(19) United States
(12) Patent Application Publication
(10) Pub. No.: US 2016/0213684 A1
(43) Pub. Date: Jul. 28, 2016

(54) SOLID DISPERSION OF A SELECTIVE MODULATOR OF THE PROGESTERONE RECEPTOR

(71) Applicant: LABORATOIRE HRA-PHARMA, Paris (FR)

(72) Inventors: Florian BATTUNG, Paris (FR); Pierre-Yves JUVIN, Nantes (FR); Jérôme HECQ, Camblanes Et Meynac (FR); Aude COLIN, Bordeaux (FR)

(21) Appl. No.: 14/649,296

(22) PCT Filed: Dec. 5, 2013
(86) PCT No.: PCT/FR2013/052952

§ 371 Date: Jun. 3, 2015

(30) Foreign Application Priority Data
Dec. 6, 2012 (FR) 1261721

(51) Int. Cl. A61K 31/57; A61K 9/20; A61K 9/48; A61K 9/00; A61K 9/16

(52) U.S. Cl. CPC A61K 31/57; A61K 9/0053; A61K 9/1694; A61K 9/1641; A61K 9/1635; A61K 9/4866; A61K 9/2027; A61K 9/1617

(57) ABSTRACT

The subject of the present invention is a solid dispersion comprising an active ingredient selected from the group consisting of selective progesterone receptor modulators, metabolites thereof and mixtures thereof, and a pharmaceutically acceptable polymeric excipient. The invention also relates to a pharmaceutical composition comprising said solid dispersion and to the therapeutic uses thereof.

### FIELD OF THE INVENTION

The present invention relates to a novel galenic form of a selective progesterone receptor modulator (SPRM), more specifically to a solid dispersion, and to pharmaceutical compositions containing said galenic form.

### TECHNICAL BACKGROUND OF THE INVENTION

Ulipristal acetate (abbreviated as UPA) corresponds to 17α-acetoxy-11β-[4-(N,N-dimethylamino)phenyl]-19-norpregna-4,9-diene-3,20-dione (IUPAC nomenclature) and has the following chemical formula:

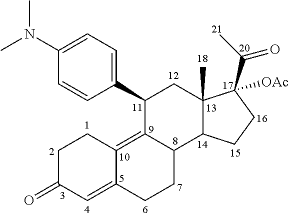 [SMILES: CC(=O)O[C@]1(C(C)=O)CCC2C3CCC4=CC(=O)CCC4=C3[C@@H](c3ccc(N(C)C)cc3)C[C@@]21C]

Its synthesis is described, inter alia, in patent EP 0 422 100 and in patent application EP 1 602 662.

Ulipristal acetate is a synthetic selective progesterone receptor modulator (SPRM). By virtue of its action on the progesterone receptor, ulipristal acetate is capable of exerting a contraceptive action by inhibiting or delaying ovulation. Clinical studies have shown that ulipristal acetate, administered in a single dose of 30 mg, makes it possible to prevent an unwanted pregnancy when it is administered within 120 hours following unprotected or poorly protected sexual intercourse (Glasier et al, Lancet. 2010, 375(9714):555-62; Fine et al, Obstet Gynecol. 2010, 115:257-63). Ulipristal acetate has thus been authorized as an emergency contraceptive and is marketed under the trade name EllaOne® in Europe.

Other therapeutic applications of ulipristal acetate have been proposed in the prior art. Recent clinical trials have shown that the chronic administration of ulipristal acetate (at 5 mg or 10 mg per day) makes it possible to significantly reduce the symptoms associated with uterine fibromas and provides a therapeutic benefit which is greater than that of the reference treatment, namely leuprolide acetate (Donnez et al., N Engl J Med. 2012; 366(5):421-32.). On the basis of these clinical trials, the European Medicines Agency (EMA) authorized, in February 2012, the speciality product Esmya® (5 mg of ulipristal acetate) for the pre-operative treatment of symptoms associated with uterine fibromas.

The pharmaceutical compositions currently marketed comprise ulipristal acetate in a micronized form.

The speciality product Esmya® is provided in the form of an uncoatedtablet comprising 5 mg of micronized ulipristal acetate combined with the following excipients: microcrystalline cellulose, mannitol, sodium croscarmellose, talc and magnesium stearate.

EllaOne® is, for its part, provided in the form of an uncoated tablet comprising 30 mg of micronized ulipristal acetate and the following excipients: lactose monohydrate, povidone K30, sodium croscarmellose and magnesium stearate.

Analogous pharmaceutical compositions have also been described in international application WO 2010/066749.

The development of new galenic forms suitable for the administration of selective progesterone receptor modulators such as ulipristal acetate remains a major challenge for their therapeutic and contraceptive uses.

In this regard, there is, at the current time, a need for alternative pharmaceutical formulations comprising a selective progesterone receptor modulator, such as ulipristal acetate, and having suitable release properties and a suitable bioavailability.

### SUMMARY OF THE INVENTION

A subject of the invention is a solid dispersion comprising:
an active ingredient selected from the group consisting of selective progesterone receptor modulators, metabolites thereof and mixtures thereof, and
a pharmaceutically acceptable polymeric excipient, preferably selected from the group consisting of polyethylene glycols, N-vinylpyrrolidone polymers and copolymers, polyacrylic acid, polymethacrylic acid, copolymers based on acrylic acid, on methacrylic acid or on esters thereof, cellulose and cellulose derivatives.

The active ingredient is preferably selected from the group consisting of 17α-acetoxy-11β-[4-N-methylaminophenyl]-19-norpregna-4,9-diene-3,20-dione, 17α-acetoxy-11β-[4-aminophenyl]-19-norpregna-4,9-diene-3,20-dione, ulipristal acetate and mixtures thereof.

In one additional embodiment, the pharmaceutically acceptable polymeric excipient is selected from the group consisting of polyvinylpyrrolidones, N-vinyl-2-pyrrolidone copolymers, and mixtures thereof.

The solid dispersion according to the invention can be characterized by one or more of the following characteristics:
the active ingredient is ulipristal acetate,
the polymeric excipient is selected from the group consisting of polyvinylpyrrolidones, copolymers of N-vinyl-2-pyrrolidone and of vinyl acetate, and mixtures thereof,
the “polymeric excipient/active ingredient” weight ratio is included in a range from 1 to 50, preferably from 1 to 30,
the solid dispersion also comprises a surfactant, it being possible for the “active ingredient/surfactant” weight ratio to be included in a range from 0.5 to 10 and/or it being possible for said surfactant to be a dodecyl sulphate salt, preferably sodium dodecyl sulphate, and
the solid dispersion is present in the form of a deposit at the surface of a pharmaceutically acceptable carrier.

A subject of the present invention is also a method for preparing a solid dispersion as previously described, characterized in that it comprises the steps consisting in:
a) preparing a solution comprising the polymeric excipient and the active ingredient in a solvent, then eliminating said solvent so as to obtain the solid dispersion; or
b) preparing a mixture comprising the polymeric excipient in the molten state and the active ingredient, then solidifying said mixture so as to obtain the solid dispersion.

An additional subject according to the invention is a pharmaceutical composition comprising a solid dispersion as previously defined and a pharmaceutically acceptable excipient, preferably selected from the group consisting of a diluent, a binder, a flow agent, a lubricant, a disintegrant and mixtures thereof. Preferably, the pharmaceutical composition comprises from 1 mg to 100 mg, preferably from 1 mg to 40 mg, of active ingredient per dose unit and is suitable for oral administration. It may be in the form of a powder, a granule, a coated or uncoated tablet, or a capsule.

A subject of the present invention is also a solid dispersion or a pharmaceutical composition, as previously described, for use as a contraceptive, in particular as an emergency contraceptive or a regular contraceptive.

Finally, a subject of the invention is also a solid dispersion or a pharmaceutical composition, as previously described, for use in the treatment or prevention of a gynaecological disorder, preferably affecting the uterus, for example uterine fibromas.

### FIGURES

FIG. 1 shows the in vitro dissolution curves for various solid dispersions obtained by means of the “solvent evaporation” method (see Exemple 1 hereinafter): UPA/povidone 3/7 (solid square), UPA/copovidone 3/7 (open circle), Control experiment: micronized UPA (solid triangle). y-axis: percentage of UPA released (%), x-axis: time in minutes.

FIG. 2 shows the in vitro dissolution curves for various solid dispersions (see Example 1 hereinafter): UPA/copovidone/SDS 3/6/1 (solid square), UPA/povidone/SDS 3/6/1 (solid triangle), UPA/povidone/SDS 3/5/2 (cross), Control experiment: micronized UPA (open circle). y-axis: percentage of UPA released (%), x-axis: time in minutes.

FIG. 3 shows the X-ray powder diffraction spectra of:
non-micronized UPA (curve 1),
the solid dispersion UPA/povidone/SDS 3/5/2 before (curve I) and after storage for 1 month (curve II) and
the solid dispersion UPA/povidone/SDS 3/5/1 before (curve a) and after storage for 1 month (curve b).

FIG. 4 shows the in vitro dissolution curves for various solid dispersions obtained by means of the melting method (see Example 1 hereinafter): UPA/PEG-4000 (open diamond), UPA/PEG-1450/SDS (open triangle), UPA/Gelucire® (solid circle), Control experiment: micronized UPA (solid square). y-axis: percentage of UPA released (%), x-axis: time in minutes.

### DETAILED DESCRIPTION OF THE INVENTION

### Solid Dispersion According to the Invention and Preparation Process

The Applicant showed that it is possible to significantly improve the in vitro dissolution profile of ulipristal acetate (hereinafter UPA) by virtue of a solid dispersion technology. In this regard, Example 1 of the present application shows that a solid dispersion comprising ulipristal acetate dispersed in a polymeric matrix, for example of povidone or copovidone type, has in vitro dissolution properties for UPA—in particular a dissolution rate—much better than those of micronized UPA. This improvement in the UPA in vitro dissolution properties provided by the solid dispersion technique should result, in vivo, in a clear increase in UPA bioavailability. The solid dispersion is expected to make it possible to decrease the dose of UPA to be administered to the patient in order to obtain the desired therapeutic or contraceptive effect, thus improving the safety, in particular the innocuousness, of UPA-based pharmaceutical compositions.

Without wishing to be bound by any theory, the Applicant is of the opinion that, in the very specific context of the present application, the results obtained for UPA can be extrapolated to other selective progesterone receptor modulators.

Thus, a first subject of the present invention is a novel galenic form, more specifically a solid dispersion comprising:
a polymeric excipient, and
an active ingredient selected from the group consisting of selective progesterone receptor modulators, metabolites thereof and mixtures thereof.

The term “solid dispersion” is intended to mean a system in the solid state at ambient temperature, comprising particles of active ingredient which are dispersed, preferably homogeneously, in a solid matrix. In the context of the present invention, the solid matrix comprises an excipient of the polymeric type, i.e. a pharmaceutically acceptable polymer or a mixture of pharmaceutically acceptable polymers. The solid dispersion may comprise, in addition to the active ingredient and the polymeric excipient, one or more additional excipients, which are either a constituent of the matrix, or are dispersed in said matrix. The solid dispersion can be obtained by any method known to those skilled in the art. The present description provides, further below, several methods suitable for preparing a solid dispersion according to the invention.

The term “polymeric excipient” is intended to mean any polymer or mixture of polymers suitable for use as a solid-dispersion matrix and which are pharmaceutically acceptable.

The polymeric excipient may be selected from:
polymers having a glass transition temperature (Tg) below 200° C., preferably below 100° C., and mixtures thereof, and
polymers which are soluble in water or in an organic solvent, said solvent possibly being dichloromethane, acetone, an alcohol, for example methanol, ethanol or isopropanol, or else an ester such as ethyl acetate.

The polymeric excipients suitable for use as a constituent of a solid-dispersion matrix comprise, without being limited thereto, polyethylene glycols, N-vinylpyrrolidone polymers and copolymers, polyacrylic acid, polymethacrylic acid, copolymers based on acrylic acid, on methacrylic acid or on esters thereof, for example methyl or ethyl esters thereof, cellulose and cellulose derivatives.

Suitable polyethylene glycols generally have an average molecular weight included in a range from approximately 150 to 35 000 g·mol⁻¹, preferably from approximately 300 to approximately 10 000 g·mol⁻¹, such as PEG 200, PEG 400, PEG 1450 or PEG 4000. The cellulose derivatives encompass, without being limited thereto, cellulose esters and ethers, such as cellulose acetate, cellulose phthalate acetate or hydroxypropylmethylcellulose.

In some embodiments, the polymeric excipient is selected from the group consisting of polyethylene glycols, N-vinyl-2-pyrrolidone polymers and copolymers, copolymers and polymers based on acrylic acid, on methacrylic acid or on esters thereof, and mixtures of said polymers and copolymers.

In a preferred embodiment, the polymeric excipient is selected from the group consisting of N-vinyl-2-pyrrolidone polymers and copolymers, copolymers and polymers based on acrylic acid, on methacrylic acid or on esters thereof, and mixtures of said polymers and copolymers. As is illustrated in the examples, the N-vinylpyrrolidone-based polymers have a high active ingredient load capacity.

Thus, in one even more preferred embodiment, the polymeric excipient is selected from the group consisting of N-vinyl-2-pyrrolidone polymers and copolymers, and mixtures thereof. Preferably, these polymers are non-crosslinked. In other words, said polymers preferably do not encompass crospovidones.

The N-vinyl-2-pyrrolidone polymers correspond to N-vinylpyrrolidone homopolymers, also known in the literature as polyvinylpyrrolidone, povidone or PVP. Polyvinylpyrrolidones suitable for implementing the present invention can have a molecular weight ranging from 10³ to 10⁷ g·mol⁻¹, preferably from 10 000 to 300 000 g·mol⁻¹, and even more preferably from 10 000 to 100 000 g·mol⁻¹. The polyvinylpyrrolidones suitable for implementing the invention are, for example, polyvinylpyrrolidones which are soluble in water or in an alcohol.

An example of suitable PVP is the PVP sold under the name Plasdone® K29-32 by the company ISP, which has an average molecular weight of approximately 58 000 g·mol⁻¹ and a K-value ranging from 29 to 32.

The N-vinyl-2-pyrrolidone copolymers correspond to polymers comprising at least two distinct types of monomers, including monomers of N-vinyl-2-pyrrolidone type. The N-vinyl-2-pyrrolidone copolymers encompass, for example, copolymers of N-vinyl-2-pyrrolidone and of vinyl acetate. The copolymers of N-vinyl-2-pyrrolidone and of vinyl acetate are also denoted, in the prior art, by the term copovidone. A copovidone suitable for implementing the present invention may comprise a molar ration of N-vinyl-2-pyrrolidone to vinyl acetate ranging from 3/7 to 7/3, preferably from 5/5 to 7/3, for example a molar ratio of about 6/4.

The term “selective progesterone receptor modulator” is intended to mean a progesterone receptor ligand which exerts an agonist activity, an antagonist activity or a mixed agonist/antagonist activity in a tissue-specific manner, preferably an agonist or a mixed agonist/antagonist activity. By virtue of their general knowledge, those skilled in the art will be able to determine, by means of routine experiments, whether a compound is an SPRM, in particular by referring to the articles by Smith and O'Malley, Endocrine Review, 25(1):45-71, and by Chabbert-Buffet et al., Human Reproduction Update, 2005, 11, 293-307. Preferably, the SPRM compound is a steroidal derivative. Examples of steroidal SPRMs are provided in the following publications: Rao et al., Steroids, 1998, 63:523-530 and Chabbert-Buffet et al., Human Reproduction Update, 2005, 11, 293-307. In particular, Chabbert-Buffet et al. mention mifepristone, onapristone, asoprisnil, ulipristal acetate, Org 33628 and Org 31710 as being SPRMs. The specific progesterone receptor modulators are preferably steroidal derivatives substituted in the 1113 position with an aryl group.

Thus, in one advantageous embodiment, the SPRM(s) present in the solid dispersion is (are) selected from the compounds of formula (I) below:

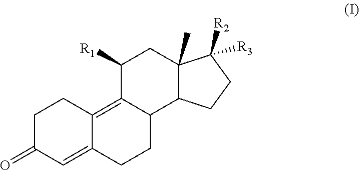

in which:
R₁ represents an aryl group optionally substituted with one or more groups independently in the ortho, para or meta position,
R₂ represents —OH, a C₁-C₅ alkoxy group or —C(O)—R₄, and
R₃ represents —OH, a C₁-C₅ alkoxy group, a C₂-C₅ alkynyl group, a C₂-C₅ alkenyl group or —O—C(O)—R₅,
R₄ and R₅ being selected, independently of one another, from a C₁-C₃ alkyl group and a C₁-C₅ alkoxy group, and also the pharmaceutically acceptable salts thereof.

A C₁-C₃ alkyl encompasses methyl, ethyl, propyl and isopropyl groups.

A C₁-C₅ alkoxy group encompasses the groups of formula —(CH₂)ₙO(CH₂)_{(y-n)}CH₃, n being an integer ranging from 0 to 4, y being an integer from 0 to 4, it being understood that (y-n) is greater than or equal to 0.

In one preferred embodiment, the SPRM is selected from the group of SPRM compounds of formula (Ia) below:

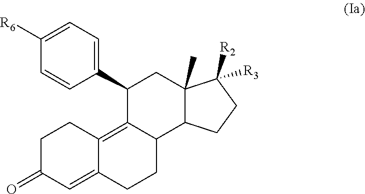

in which:
R₆ represents:
   —NR₇R₈ in which R₇ and R₈ represent, independently of one another, —H or a C₁-C₃ alkyl, R₇ and R₈ preferably being selected from H and —CH₃;
   —CH═N—O—R₉ in which R₉ represents —H or —C(O)—X—R₁₀ with R₁₀ being a C₁-C₃ alkyl and X representing O, NH or S; or
   —C(O)Rᵢᵢ in which Rᵢᵢ represents a C₁-C₃ alkyl;
R₂ and R₃ being as previously defined.

Such compounds comprise, without being limited thereto, mifepristone, ulipristal acetate, asoprisnil and telapristone. In particular, mifepristone corresponds to the compound of formula (Ia) in which R₆ is —N(CH₃)₂, R₂ is OH and R₃ is —C≡C—CH₃.

In one preferred embodiment, the SPRM is selected from the group of SPRM compounds of formula (Ia) in which:
R₂ represents —OH, —OCH₃, —C(O)—CH₃ or —C(O)—CH₂—O—CH₃
R₃ represents —CH₂—O—CH₃, or —O—C(O)—CH₃
R₆ represents —NH₂, —NH—CH₃, —N(CH₃)₂ or —CH═N—OR₉ in which R₉ represents H, —C(O)—S—C₂H₅ or —C(O)—NH—C₂H₅.

This group of compounds encompasses, inter alia, telapristone, asoprisnil and ulipristal acetate, and certain metabolites thereof.

In certain embodiments, the SPRM is selected from the compounds of formula (Ia) in which R₆ is —CH═N—O—R₉. Such compounds encompass:
Asoprisnil (R₉ is H, R₂ is OMe and R₃ is —CH₂OMe),
J912 (R₉ is H, R₂ is H and R₃ is —CH₂OMe),
J956 (also known as Asoprisnil ecamate) (R₉ is —C(O)—NH—C₂H₅, R₂ is —OMe and R₃ is —CH₂OMe), and
J1042 (R₉ is —C(O)—S—C₂H₅, R₂ is —OCH₃ and R₃ is —CH₂OMe).

In other embodiments, the SPRM is selected from the compounds of formula (Ia) in which R₆ is —NH₂, —NHCH₃, or —N(CH₃)₂.

In a preferred embodiment, the active ingredient present in the solid dispersion is selected from the group consisting of ulipristal acetate, ulipristal acetate metabolites and mixtures thereof.

UPA metabolites are described, inter alia, in Attardi et al., Journal of Steroid Biochemistry and Molecular Biology, 2004, 88: 277-288 and illustrated hereinafter:

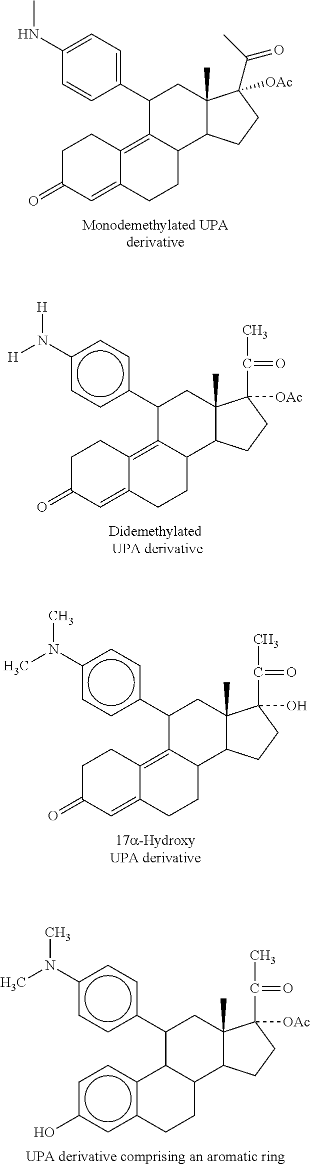

Preferably, the ulipristal acetate metabolite is selected from:
17α-acetoxy-11β-[4-N-methylaminophenyl]-19-norpregna-4,9-diene-3,20-dione (monodemethylated derivative) and
17α-acetoxy-11β-[4-aminophenyl]-19-norpregna-4,9-diene-3,20-dione (didemethylated derivative).

In one preferred embodiment, the active ingredient present in the solid dispersion is selected from the group consisting of 17α-acetoxy-11β-[4-N-methylaminophenyl]-19-norpregna-4,9-diene-3,20-dione, 17α-acetoxy-11β-[4-aminophenyl]-19-norpregna-4,9-diene-3,20-dione, ulipristal acetate and mixtures thereof.

In an additional embodiment, the solid dispersion comprises:
an active ingredient selected from the group consisting of 17α-acetoxy-11β-[4-N-methylaminophenyl]-19-norpregna-4,9-diene-3,20-dione, 17α-acetoxy-11β-[4-aminophenyl]-19-norpregna-4,9-diene-3,20-dione, ulipristal acetate and mixtures thereof, preferably ulipristal acetate, and
a polymeric excipient selected from the group consisting of polyethylene glycols, N-vinylpyrrolidone polymers and copolymers, polyacrylic acid, polymethacrylic acid, copolymers based on acrylic acid, on methacrylic acid or on esters thereof, cellulose and cellulose derivatives, and combinations thereof.

Preferably, the polymeric excipient is selected from the group consisting of polyvinylpyrrolidones, N-vinylpyrrolidone copolymers, such as copovidones, and combinations thereof.

The weight ratio between the polymeric excipient and the active ingredient in the solid dispersion according to the invention is generally in a range from 1 to 100, preferably from 1 to 80, more preferably from 1 to 60, and even more preferably from 1 to 50. A weight ratio in a range of from 1 to 50 encompasses a weight ratio in a range from 1 to 10, from 10 to 20, from 20 to 25, from 25 to 30, from 30 to 40, and from 40 to 50. In particular, the weight ratio may be included in a range from 1 to 30.

In certain embodiments, the “polymeric excipient/active ingredient” weight ratio is included in a range from 1 to 20, preferably from 1.5 to 10, or even from 1.5 to 5. Such weight ratios can be obtained, for example, for a solid dispersion prepared by means of the “solvent evaporation” method.

In other embodiments, the “polymeric excipient/active ingredient” weight ratio is included in a range from 20 to 50, preferably from 20 to 30. Such a weight ratio can be obtained, for example, for solid dispersions prepared by means of the “melting” method.

The methods for preparing the solid dispersions by melting and by solvent evaporation are described later on in the present description.

The “polymeric excipient/active ingredient” weight ratio may be selected according to the final use thereof. By way of example, when the solid dispersion is integrated into an oral tablet, the “polymeric excipient/active ingredient” weight ratio is selected in such a way that the size of the final tablet allows it to be administered to the patient orally.

In certain embodiments, the solid dispersion according to the invention may comprise an additional excipient. This additional excipient can make it possible to potentiate the action of the polymeric excipient on the dissolution properties or to improve the bioavailability of ulipristal acetate. This additional excipient may be a constituent of the matrix of the solid dispersion or may be dispersed therein. In certain embodiments, this excipient is selected from pharmaceutically acceptable surfactants. Such surfactant encompass, without being limited thereto, simethicone, triethanolamine, polysorbate derivatives such as Tween® 20 or Tween® 40, poloxamers, fatty alcohols such as lauryl alcohol or cetyl alcohol, or else alkyl sulphates.

The surfactant may in particular be selected from C₈-C₂₀ alkyl sulphate salts, preferably C₁₀-C₁₄ alkyl sulphate salts, and mixtures thereof. Preferably, the surfactant is selected from the dodecyl sulphate salts, preferably the alkali metal or alkaline-earth metal salts thereof, such as a sodium, magnesium or calcium salt. A particularly preferred surfactant is SDS, i.e. sodium dodecyl sulphate.

By way of example, the solid dispersion according to the invention may comprise:
an active ingredient selected from the group consisting of ulipristal acetate, 17α-acetoxy-11β-(4-N-methylaminophenyl)-19-norpregna-4,9-diene-3,20-dione, 17α-acetoxy-11β-(4-aminophenyl)-19-norpregna-4,9-diene-3,20-dione, and mixtures thereof, preferably ulipristal acetate.
a polymeric excipient selected from povidones, copovidones and mixtures thereof, and
a solid surfactant, preferably SDS.

The weight ratio between the active ingredient and the surfactant is generally in a range from 0.5 to 10, preferably from 0.5 to 4. A suitable “active ingredient/surfactant” weight ratio is, for example, a weight ratio of about 3.

In other embodiments, the solid dispersion is free of any surfactant and more generally of any excipient in addition to the polymeric excipient. Thus, in certain embodiments, the solid dispersion may consist of solely the active ingredient and the polymeric excipient.

In certain embodiments, the active ingredient is present predominantly in an amorphous form in the solid dispersion according to the invention.

The expression “active ingredient present predominantly in an amorphous form” is intended to mean the fact that at least 80% of the active ingredient present in the solid dispersion is in amorphous form. In other words, the solid dispersion contains at most 20% of active ingredient in crystalline form, the percentages referring to the total amount of active ingredient present in the solid dispersion.

The active ingredient may be present essentially in an amorphous form in the solid dispersion according to the invention. In other words, at least 95% of the active ingredient present in the solid dispersion may be in amorphous form. “At least 95% of active ingredient in amorphous form” encompasses at least 96%, at least 97%, at least 98%, at least 98.5%, at least 99%, at least 99.2%, at least 99.44%, at least 99.6%, at least 99.8%, and at least 99.9% of active ingredient in amorphous form.

In order for the active ingredient to be present predominantly in an amorphous form, it is preferable for the solid dispersion to comprise less than 20% by weight of surfactant, preferably less than 15% by weight of surfactant, or even at most 10% by weight of surfactant.

Depending on its preparation mode, the solid dispersion according to the invention may be in the form of a powder, optionally micronized, or in the form of a deposit on a pharmaceutically acceptable carrier. The pharmaceutically acceptable carrier may be a diluent such as microcrystalline cellulose or monosaccharides such as lactose or mannitol. Additional examples of diluents are provided later on in the present description.

The term “micronized powder” is intended to mean a powder of which the particles size is characterized by a d90 of less than 50 μm, which means that at least 90% of the particles of the powder have a size of less than 50 μm, the size of the particles referring to their largest dimension.

As is shown in the examples, the solid dispersion according to the invention has improved properties of in vitro dissolution of the active ingredient. In certain embodiments, the solid dispersion according to the invention is characterized in that at least 60% of the active ingredient that it contains is released within 45 minutes when said solid dispersion is subjected to an in vitro dissolution test.

The in vitro dissolution test can be carried out using any commercially available dissolution device comprising paddles. Example 1 hereinafter presents implementing conditions for determining the in vitro dissolution rate of a solid dispersion according to the invention. Briefly, an amount of solid dispersion representing 30 mg of active ingredient is placed in a gelatin capsule. This gel capsule is then placed in 900 ml of a medium buffered at gastric pH, comprising 0.1% of SDS, at 37±0.5° C., and subjected to stirring at 50 revolutions per minute (rpm) (speed of rotation of the paddles of the dissolution device). The dissolution of the active ingredient in the medium can be monitored by spectrophotometry, preferably at the maximum wavelength of absorbance. A gastric pH is typically a pH of 1 to 3.

“At least 60% of the active ingredient released within 45 minutes” encompasses at least 65%, at least 70%, and at least 75% of the active ingredient is released within 45 minutes. Preferably, at least 65% of the active ingredient is released within 45 minutes.

The solid dispersion can also be characterized in that at least 55% of the active ingredient that it contains is released within 30 minutes when it is subjected to an in vitro dissolution test as previously described.

The solid dispersion can be obtained by means of any one of the methods described in the prior art. In particular, the solid dispersion can be obtained by means of a first method termed “solvent evaporation method” comprising the preparation of a solution containing the polymeric excipient, the active ingredient and, optionally, the additional pharmaceutical excipient, in a solvent, and then the elimination of said solvent so as to obtain the solid dispersion.

The solvent is selected in such a way as to dissolve the polymeric excipient, the active ingredient and the optional additional excipient. The elimination of the solvent can be carried out by means of any known method, for example by evaporation through heating, by evaporation under reduced pressure or under vacuum, by lyophilization or by spray-drying. Spray-drying consists in dispersing the solution in the form of fine droplets in hot air by means of nozzles. The final size of the particles of the solid dispersion depends on the size of the droplets formed. In certain cases, the solution is vaporised on particles of a pharmaceutically acceptable carrier, typically a diluent, said carrier particles being suspended in hot air within a fluidized bed. The final solid dispersion is then in the form of a deposit at the surface of the diluent particles.

Lyophilization, for its part, comprises the freezing of the solution and then the elimination of the solvent by sublimation.

By way of alternative, the solid dispersion can be obtained by means of a second method termed “melting method” in which a mixture, comprising the polymeric excipient and the active ingredient, in the molten state is prepared and then solidified so as to obtain the solid dispersion. The mixture may also comprise an additional pharmaceutical excipient.

The mixture may be formed according to several methods. For example, a mixture containing the polymeric excipient and the active ingredient (and optionally the additional excipient) is prepared and then this mixture is heated with stirring so as to promote melting of the polymeric excipient. By way of alternative, the polymeric excipient may firstly be heated so as to melt it, and then the active ingredient and optionally the additional pharmaceutical excipient are added, with stirring. The heating temperature is generally above the glass transition temperature (Tg) of the polymeric excipient. Preferably, the active ingredient and the optional additional excipient are soluble in the molten polymeric excipient.

The mixture can be prepared in a conventional reactor equipped with a heating means and optionally with a cooling means. By way of alternative, an extruder equipped with a heating means may be used. The use of an extruder coupled to a means for cutting up or for moulding the extrudate makes it possible to obtain dosage forms, for example tablets, directly at the output of the process, without a subsequent shaping step.

The solidification of the solid dispersion can be carried out by allowing the mixture comprising the molten polymeric excipient and the active ingredient to return to ambient temperature, or by cooling it. The solidification can also be obtained by spray-drying if the viscosity of the mixture allows it.

Finally, the solid dispersion can be prepared by means of a method based on the use of a supercritical fluid, for example supercritical CO2. The solid dispersion can be prepared by means of a process known as “rapid expansion of supercritical solution (RESS)”. In this process, the supercritical fluid is used as solvent to dissolve the active ingredient and the polymeric excipient (and the optional additional excipient). This supercritical solution is then spray-dried in an expansion chamber at low pressure. The low pressure present in the expansion chamber results in the formation of particles of solid dispersion. This technique is conceptually close to the “solvent” method, the supercritical fluid replacing the liquid solvent.

By way of alternative, the supercritical fluid can be used as anti-solvent. In this case, a solution containing the polymeric excipient and the active ingredient (and the optional additional excipient) is sprayed in a chamber containing a supercritical fluid. The diffusion of the supercritical fluid in the droplets brings about the precipitation of the solid dispersion. Such a process is generally known as “Precipitation with Compressed Anti-solvent (PCA)”. In a preferred embodiment, the solid dispersion is prepared by means of the solvent evaporation method or by means of the melting method, as previously described. Thus, a subject of the present invention is also a method for preparing a solid dispersion comprising the steps consisting in:
a) preparing a solution comprising the polymeric excipient and the active ingredient in a solvent, then eliminating said solvent so as to obtain the solid dispersion; or
b) preparing a mixture comprising the polymeric excipient in the molten state and the active ingredient, then solidifying said mixture so as to obtain the solid dispersion.

The alternative a) corresponds to the solvent method of preparation, whereas the alternative b) corresponds to the melting method of preparation.

The solvent evaporation method (alternative a) is generally used when there is a solvent capable of dissolving both the active ingredient and the polymeric excipient, and, if present, the additional excipient.

In this case, the polymeric excipient can be selected from the group consisting of polyvinylpyrrolidones, N-vinylpyrrolidone copolymers (for example copovidones) and combinations thereof. The solvent may be acetone, an alcohol, for example methanol, ethanol, isopropanol, 2-methyl-2-propanol, isobutanol or tert-butanol, dichloromethane, chloroform, or else an ester such as ethyl acetate, or a mixture thereof. The “polymeric excipient/active ingredient” weight ratio in a solid dispersion obtained by means of the solvent evaporation method is generally from 1 to 20.

The melting method (alternative b) is, for its part, preferably used when the polymeric excipient has a low glass transition temperature—typically below 100° C. By way of example, the solid dispersion can be prepared by means of the “melting” method when the polymeric excipient is a polyethylene glycol. The “polymeric excipient/active ingredient” weight ratio in a solid dispersion obtained by means of the melting method may be from 20 to 50, preferably from 20 to 30.

The method for preparing the solid dispersion according to the invention may comprise additional steps, such as steps of milling, sieving or else moulding the solid dispersion.

### Pharmaceutical Composition According to the Invention

The solid dispersion according to the invention is intended mainly for therapeutic or contraceptive use. For this purpose, it can be administered directly or inserted into an administration device such as a vaginal ring, a patch, an intra-uterine device or an implant. The solid dispersion according to the invention may also be integrated into a pharmaceutical composition. Thus, an additional subject of the present invention is a pharmaceutical composition comprising a solid dispersion as previously defined and at least one pharmaceutically acceptable excipient. Those skilled in the art will be able to choose the excipient(s) to be combined with the solid dispersion according to the final form of the pharmaceutical composition, the desired route of administration and the desired active ingredient release profile. For this purpose, those skilled in the art will be able to refer to the following reference works: Remington: The Science and Practice of Pharmacy (Lippincott Williams & Wilkins; Twenty first Edition, 2005), and Handbook of Pharmaceuticals Excipients, American Pharmaceutical Association (Pharmaceutical Press; 6th revised edition, 2009). The pharmaceutical composition and the solid dispersion according to the invention may be administered by any route, in particular by the oral, buccal, nasal, sublingual, vaginal, intra-uterine, rectal or transdermal route or by the parenteral route, for example by intravenous injection. The preferred routes of administration are the buccal, oral, intra-uterine and vaginal routes. The pharmaceutical composition according to the invention may be in any form, for example in the form of a tablet, a powder, a capsule, a pill, a suppository, a pessary, a suspension, a syrup, a gel, an ointment, an emulsion, a lyophilisate or an orodispersible film. The route of administration and the galenic form of the pharmaceutical composition may depend on the desired therapeutic or contraceptive effect. In certain embodiments, the pharmaceutical composition according to the invention may be integrated into a device enabling prolonged administration of the active ingredient. The pharmaceutical composition may in particular be integrated into a vaginal ring, into a patch, for example a transdermal or mucoadhesive patch, into an intra-uterine device or into an implant, for example an implant of contraceptive type. For examples of vaginal rings suitable for implementing the invention, reference may be made to application WO 2006/10097.

In additional embodiments, the pharmaceutical composition according to the invention is in solid form. Preferably, the pharmaceutical composition according to the invention is solid and is intended for oral administration.

In certain embodiments, the pharmaceutical composition according to the invention is characterized in that the pharmaceutically acceptable excipient is selected from the group consisting of a diluent, a binder, a flow agent, a lubricant, a disintegrant and mixtures thereof. For the purposes of the present invention, a diluent may be one or more compounds capable of densifying the active ingredient (in this case the solid dispersion) so as to obtain the desired mass. The diluants encompass inorganic phosphates, monosaccharides and polyols such as xylitol, sorbitol, lactose, galactose, xylose or mannitol, disaccharides such as sucrose, oligosaccharides, polysaccharides such as cellulose and its derivatives, starches, and mixtures thereof. The diluent may be in anhydrous or hydrated form. By way of example, the diluent may be selected from microcrystalline cellulose, mannitol, lactose and mixtures thereof.

The binder may be one or more compounds capable of improving the aggregation of the active ingredient with the diluent. By way of example, mention may be made of hydroxypropylcellulose, hydroxypropylmethylcellulose, povidone (polyvinylpyrrolidone), copolymers of N-vinyl-2-pyrrolidone and of vinyl acetate (copovidone), and mixtures thereof.

The lubricant may be one or more compounds capable of preventing the problems associated with the preparation of dry galenic forms, such as the sticking and/or gripping problems which occur in machines during compression or filling. The preferred lubricants are fatty acids or fatty acid derivatives, such as calcium stearate, glyceryl monostearate, glyceryl palmitostearate, magnesium stearate, zinc stearate, or stearic acid, polyalkylene glycols, in particular polyethylene glycol, sodium benzoate or talc. The lubricants that are preferred according to the invention are the stearate salts and mixtures thereof. A suitable lubricant is, for example, magnesium stearate.

The flow agent optionally used may be selected from compounds which contain silicon, for example talc, anhydrous colloidal silica or precipitated silica.

The disintegrant can be used to improve the release of the active ingredient. It may be selected from crosslinked polyvinylpyrrolidone (crospovidone), crosslinked carboxymethylcellulose (such as sodium croscarmellose) or non-crosslinked carboxymethylcellulose, starches, and mixtures thereof. The disintegrant is preferably selected from the group consisting of a sodium croscarmellose, a crospovidone and mixtures thereof.

In certain embodiments, the composition according to the invention comprises:
0.5% to 95% of the solid dispersion as previously defined,
5% to 95% of diluent, and
0% to 5% of lubricant,
   the percentages being expressed by weight relative to the total weight of the composition.

The composition according to the invention may in addition be characterized in that it comprises from 0% to 20% by weight of a binder, from 0% to 10% of a disintegrant and from 0% to 5% by weight of a flow agent.

Preferably, the composition according to the invention comprises:
10% to 95% by weight of solid dispersion as previously defined,
5% to 90% by weight of diluent, and
0% to 5% by weight of lubricant.

By way of example, the pharmaceutical composition according to the invention may comprise from:
4% to 25% by weight of solid dispersion,
70% to 90% by weight of diluent,
0.5% to 8% by weight of disintegrant, and
0.1% to 5% of a lubricant,
   the percentages being expressed by weight relative to the total weight of the composition.

A further example is a pharmaceutical composition comprising from:
35% to 60% by weight of solid dispersion,
35% to 60% by weight of diluent,
0.5% to 8% by weight of disintegrant, and
0.1% to 5% of a lubricant,
   the percentages being expressed by weight relative to the total weight of the composition.

The pharmaceutical composition according to the invention may also comprise one or more excipients in addition to the abovementioned excipients. The additional excipient(s) may be selected from the group consisting of surfactants such as SDS, coating agents, such as coating agents based on polyvinyl alcohol or on hydroxypropylmethylcellulose, pigments such as aluminium oxide or iron oxide, flavourings, wetting agents, waxes, dispersants, stabilizers and preservatives.

The pharmaceutical composition according to the invention may be prepared according to any one of the methods commonly used in galenics. These methods typically comprise mixing the solid dispersion according to the invention with one or more excipients, then shaping the mixture obtained. By way of example, when it is in the form of a tablet, the pharmaceutical composition according to the invention can be prepared by direct compression or by compression after dry or wet granulation.

By way of alternative, the pharmaceutical composition can be prepared according to a process comprising a step of forming a deposit of solid dispersion according to the invention on the diluent particles, typically by vaporization.

The solid dispersion present in the pharmaceutical composition according to the invention may have any one of the characteristics described in the present description. In particular, the solid dispersion present in the pharmaceutical composition has one or more (1, 2, 3, 4, 5 or 6) of the following characteristics:
i. the active ingredient is ulipristal acetate or metabolites thereof,
ii. the polymeric excipient is selected from the group consisting of polyvinylpyrrolidones, N-vinylpyrrolidone copolymers, such as copovidones, and combinations thereof,
iii. the “polymeric excipient/active ingredient” weight ratio is included in a range from 1 to 50, preferably from 1 to 30, or even from 1 to 20 and even from 1.5 to 10,
iv. the solid dispersion also comprises a surfactant, preferably SDS, preferably in a content of less than 10% by weight relative to the total weight of the solid dispersion,
v. the active ingredient is present in the solid dispersion essentially in an amorphous form,
vi. at least 60% of the active ingredient contained in the solid dispersion is released within 45 minutes when said solid dispersion is subjected to an in vitro dissolution test, preferably under the following conditions:
   device: paddle dissolution device,
   sample: hard gelatin capsule containing an amount of solid dispersion corresponding to 30 mg of active ingredient,
   dissolution medium: 900 ml of an aqueous solution buffered at gastric pH comprising 0.1% of SDS,
   temperature: 37±0.5° C., and
   paddle rotation speed: 50 revolutions per minute (rpm).

As previously mentioned, the composition according to the invention may be in the form of a powder, a granule, a coated or uncoated tablet, or a capsule, and is preferably intended for oral administration. In certain embodiments, the pharmaceutical composition according to the invention is in the form of an uncoated tablet intended for oral administration.

The composition according to the invention may be a controlled-, immediate-, sustained- or delayed-release pharmaceutical composition. Preferably, the composition according to the invention is an immediate-release composition.

The term “immediate-release composition” is intended to mean a pharmaceutical composition characterized in that at least 60%, preferably at least 75%, of the active ingredient initially contained in a dose unit of the pharmaceutical composition is released within 45 minutes when said dose unit is subjected to an in vitro dissolution test, for example according to the European Pharmacopoeia §2.9.3, preferably under the following conditions:
paddle dissolution device,
dissolution medium: aqueous solution buffered at gastric pH containing 0.1% of SDS,
temperature: 37±0.5° C., and
rotation speed: 50 rpm.

The volume of the dissolution medium depends on the amount of active ingredient contained in the dose unit. For a dose unit comprising 5 mg of active ingredient, 500 ml of dissolution medium are used. For a dose unit comprising 30 mg of active ingredient, 900 ml of dissolution medium are used.

Generally, the pharmaceutical composition comprises from 1 mg to 100 mg of active ingredient per dose unit, preferably from 1 mg to 40 mg, or even from 2 mg to 30 mg, of active ingredient per dose unit. The dose of active ingredient depends on the therapeutic or contraceptive effect and on the administration scheme that are desired. For example, for certain applications, the amount of active ingredient per dose unit may be included in a range from 1 mg to 5 mg.

In emergency contraception, the active ingredient, preferably UPA, may be present in an amount of from 20 mg to 40 mg per dose unit. In regular contraception, the active ingredient, preferably UPA, may be present in an amount of from 1 mg to 5 mg per dose unit.

For therapeutic uses such as the treatment of uterine fibromas, the active ingredient, preferably UPA, may be present in an amount of from 3 mg to 15 mg per dose unit. The dose of active ingredient and the administration scheme may also depend on the personal parameters of the patient, in particular the weight, age, sex, general health condition and diet, on the pathological conditions from which the patient is suffering, etc.

Finally, the pharmaceutical composition according to the invention may comprise an additional active ingredient. This additional active agent may exert an action different from that of the selective progesterone receptor modulator, preferably ulipristal acetate, or its metabolites. It may also reinforce the therapeutic effect of the selective progesterone receptor modulator, preferably ulipristal acetate, or the metabolites thereof.

### Therapeutic or Contraceptive Uses of the Solid Dispersion and of the Pharmaceutical Composition According to the Present Invention

In an additional aspect, a subject of the present invention is also a solid dispersion or a pharmaceutical composition, as previously described, for use as a medicament. The solid dispersion or the composition according to the invention is particularly suitable for use as a regular contraceptive or an emergency contraceptive. They can also be used for the treatment or prevention of hormonal, gynaecological or endocrine disorders, such as Cushing's disease. The composition or the solid dispersion according to the invention can be used, in particular, in the treatment or prevention of a gynaecological disorder, preferably affecting the uterus, including benign gynaecological disorders. The gynaecological disorders encompass, without being limited thereto, uterine fibromas and symptoms thereof, adenomyosis, endometriosis, pain associated with endometrium dislocation, and excessive uterine bleeding.

An additional subject of the invention is the use of a solid dispersion according to the invention, preferably containing ulipristal acetate as active ingredient, for preparing a contraceptive or for preparing a medicament intended for the treatment or prevention of any one of the abovementioned pathological conditions.

A subject of the invention is also a method of contraception comprising the administration, to a patient, of a contraceptive dose of the solid dispersion or of the pharmaceutical composition, preferably containing ulipristal acetate as active ingredient.

The term “method of contraception” is intended to mean a method which makes it possible to prevent the occurrence of a pregnancy in a patient of child-bearing age. In the case in point, it may be a method of emergency contraception. In this case, a single dose is preferably administered to the patient within an appropriate time period after unprotected or poorly protected sexual intercourse, generally within 120 h following unprotected or poorly protected sexual intercourse. The method of contraception may also be a method of regular contraception, in which the composition or the solid dispersion is administered chronically and cyclically to the patient or continuously using a device such as an implant or a vaginal ring. By way of alternative, the method of contraception may be a method of “on demand” contraception as described in international application WO 2010/119029. Finally, a subject of the invention is also a method for treating a disease or a disorder in a patient, comprising the administration of a therapeutically effective dose of the solid dispersion or of the pharmaceutical composition, preferably containing ulipristal acetate as active ingredient, to the patient, who is preferably preferably female. The therapeutic method according to the invention preferably relates to any one of the abovementioned diseases or disorders.

It goes without saying that, for the implementation of the methods and uses described above, the solid dispersion and the pharmaceutical composition according to the invention may comprise one or more of the characteristics explained in detail in the present description.

The objective of the examples hereinafter is to illustrate the invention more fully without, however, limiting the scope thereof.

### EXAMPLES

### Example 1

### Preparation and Evaluation of Various Solid Dispersions

### 1. Preparation of Solid Dispersions According to the Invention

a) Preparation of a solid dispersion by means of the melting method

A solid dispersion comprising ulipristal acetate as active ingredient and a polyethylene glycol (PEG-4000) as polymeric excipient, with a “polymeric excipient/UPA” weight ratio of 960/40, is prepared in the following way:
the polyethylene glycol is heated until it completely melts,
micronized UPA is added to the molten PEG with stirring. The mixture is heated and kept stirring until complete dissolution of the UPA,
the mixture is brought back to ambient temperature with stirring,
the solid dispersion obtained is optionally milled or micronized so as to obtain the desired particle size distribution.

Other solid dispersions were also prepared by means of the melting method:
UPA/Gelucire® 50/13 (Gattefossé) with a weight ratio 40/960,
UPA/PEG-1450/SDS with a weight ratio 40/910/50.

b) Preparation of a Solid Dispersion by Means of the “Solvent Evaporation” Method

The following solid dispersions were prepared by means of the “solvent” method:
solid dispersion UPA/Copovidone with a weight ratio of 3/7,
solid dispersion UPA/Copovidone/SDS with a weight ratio of 3/7/1,
solid dispersion UPA/Povidone according to a weight ratio of 3/7,
solid dispersion UPA/Povidone/SDS with a weight ratio of 3/6/1,
solid dispersion UPA/Povidone/SDS with a weight ratio of 3/5/2.

The UPA is micronized ulipristal acetate. The povidone corresponds to the commercial product Plasdone® K-29-32 sold by the company ISP. The copovidone corresponds, for its part, to the product Plasdone® S630 sold by ISP.

These solid dispersions were prepared in the following way:
the polymeric excipient was dissolved in ethanol at ambient temperature;
the UPA (and optionally the SDS if present) was (were) added to this solution under stirring. The solution was kept stirring and at ambient temperature until complete dissolution of the UPA;
the solvent (ethanol) was then slowly evaporated off under reduced pressure using a rotary evaporator until the solid dispersion was obtained.

### 2. In Vitro Dissolution Profiles of the Solid Dispersions Prepared

a) Materials and Methods

For each solid dispersion obtained by means of the “solvent evaporation” method (§1.b) above), hard gelatin capsules containing an amount of solid dispersion corresponding to 30 mg of UPA per capsule were prepared.

The dissolution profiles were obtained as follows:

Each capsule containing a solid dispersion was placed in a bowl of the paddle dissolution device containing 900 ml of a dissolution medium. The dissolution medium is an aqueous solution buffered at gastric pH and comprising 0.1% by weight of SDS. The conditions for carrying out the in vitro dissolutions are the following:
Paddle rotation speed: 50 revolutions per minute (rpm);
Temperature: 37° C.±0.5° C.

The dissolution of the UPA was monitored by spectrophotometry.

By way of control experiment, a gelatin capsule containing 30 mg of micronized ulipristal acetate was used. For each solid dispersion evaluated, the dissolution experiment was reproduced 3 times.

b) Results

### Solid Dispersions Prepared by Melting

Table 1 below and FIG. 5 show the dissolution results obtained for these solid dispersions. The dissolution percentages are expressed relative to the initial amount of UPA contained in each capsule.

TABLE 1
Results of the in vitro dissolution assays for the solid dispersions
prepared by melting. Control experiment: micronized UPA
Percentages of UPA released, expressed relative to the initial amount
of UPA contained per capsule (mean values over 3 experiments)

| Time | Micronized | | | UPA/PEG- |
|---|---|---|---|---|
| (min) | UPA | UPA/PEG-4000 | UPA/Gelucire | 1450/SDS |
| 0 | 0 | 0.00 | 0.00 | 0.00 |
| 1 | 0.23 | 0.70 | 0.00 | 0.20 |
| 5 | 5.69 | 3.80 | 0.30 | 5.80 |
| 7.5 | 15.28 | 8.10 | 0.70 | 13.90 |
| 10 | 21.73 | 12.40 | 1.10 | 28.50 |
| 15 | 29.46 | 28.10 | 1.80 | 62.20 |
| 20 | 35.2 | 49.90 | 2.60 | 85.40 |
| 30 | 42.14 | 89.50 | 4.20 | 93.50 |
| 45 | 49.98 | 104.50 | 6.50 | 94.70 |
| 60 | 55.62 | 105.30 | 8.70 | 95.10 |

Notably, the solid dispersions of UPA with polymeric excipient have a much higher UPA release rate than that observed for the capsules comprising micronized UPA. On the other hand, the use of another type of solid-dispersion excipient (gelucire) has a very negative impact on the dissolution rate. This illustrates the specific effect resulting from the use of a polymeric excipient.

### Solid Dispersions Prepared by the Solvent Evaporation Method

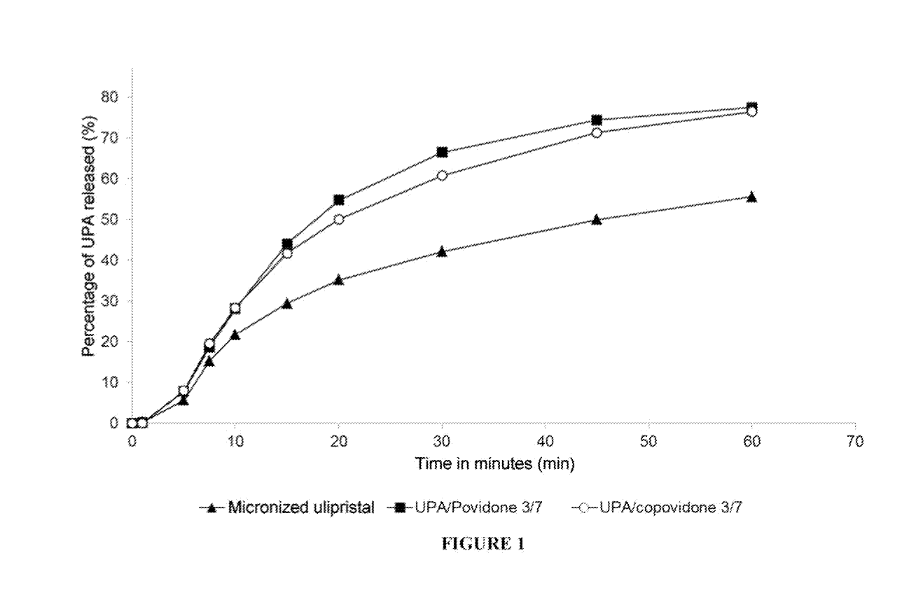

Table 2 below and FIG. 1 show the dissolution results obtained for the solid dispersions not comprising SDS. The dissolution percentages are expressed relative to the initial amount of UPA contained in each capsule.

TABLE 2
Results of the in vitro dissolution assays for the solid
dispersions prepared. Control experiment: micronized UPA

| | Percentages of UPA released, expressed | | |
|---|---|---|---|
| | relative to the initial amount of UPA contained | | |
| | per capsule (mean values over 3 experiments) | | |
| Time | | | UPA/copovidone |
| (mm) | Micronized ulipristal | UPA/Povidone 3/7 | 3/7 |
| 0 | 0 | 0.00 | 0.00 |
| 1 | 0.23 | 0.10 | 0.00 |
| 5 | 5.69 | 7.80 | 8.00 |
| 7.5 | 15.28 | 18.70 | 19.50 |
| 10 | 21.73 | 28.10 | 28.40 |
| 15 | 29.46 | 44.10 | 41.80 |
| 20 | 35.2 | 54.80 | 50.10 |
| 30 | 42.14 | 66.50 | 60.80 |
| 45 | 49.98 | 74.40 | 71.30 |
| 60 | 55.62 | 77.50 | 76.50 |

These results show that the solid dispersions of UPA with povidone or copovidone have a much higher UPA release rate than that observed for the micronized UPA. Notably, the percentage of ulipristal acetate released into the medium at t=20 min is approximately 66% for a capsule comprising the solid dispersion UPA/povidone and approximately 61% for a capsule comprising the solid dispersion UPA/copovidone, while it is only approximately 42% for a capsule containing micronized ulipristal acetate.

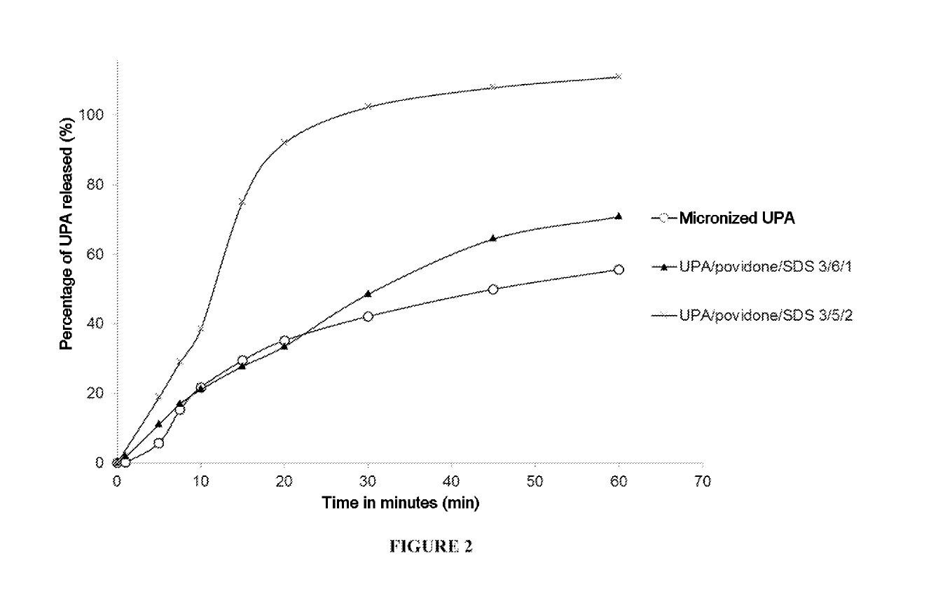

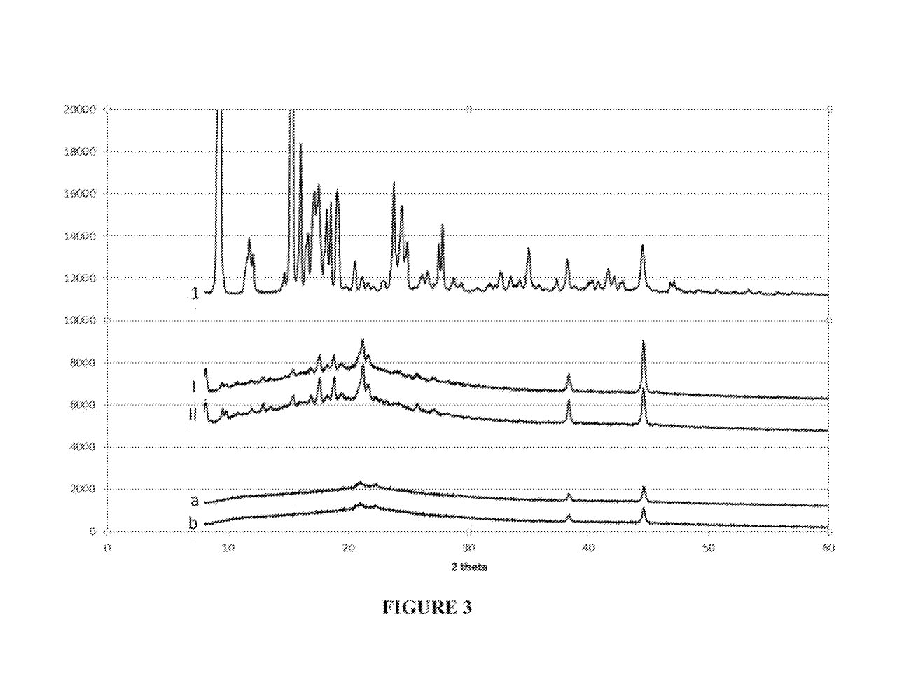

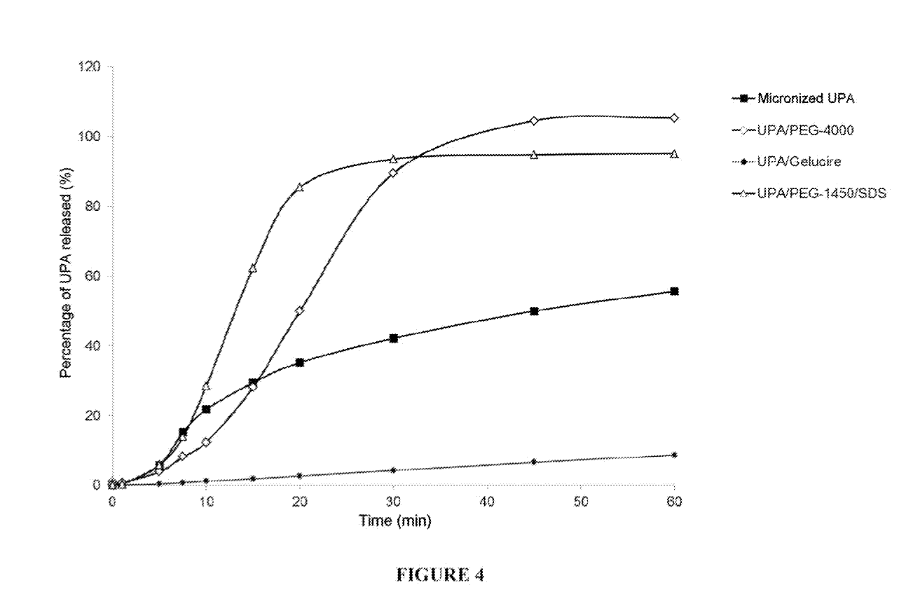

The effect of an additional excipient—namely SDS—on the UPA in vitro dissolution rate was evaluated. Table 3 below and FIG. 2 show the dissolution results obtained for the solid dispersions comprising SDS. The dissolution percentages are expressed relative to the initial amount of UPA contained in each capsule.

TABLE 3
Results of the in vitro dissolution assays for the solid
dispersions prepared. Control experiment: micronized UPA

| | Percentages of UPA released, expressed | | |
|---|---|---|---|
| | relative to the initial amount of UPA contained | | |
| | per gel capsule (mean values over 3 experiments) | | |
| Time | Micronized | UPA/povidone/SDS | |
| (min) | UPA | 3/6/1 | UPA/povidone/SDS 3/5/2 |
| 0 | 0 | 0.00 | 0 |
| 1 | 0.23 | 1.80 | — |
| 5 | 5.69 | 11.10 | 18.80 |
| 7.5 | 15.28 | 16.90 | 28.90 |
| 10 | 21.73 | 21.10 | 38.40 |
| 15 | 29.46 | 27.80 | 75.00 |
| 20 | 35.2 | 33.50 | 92.00 |
| 30 | 42.14 | 48.50 | 102.30 |
| 45 | 49.98 | 64.40 | 107.90 |
| 60 | 55.62 | 70.80 | 111.00 |

The solid dispersions UPA/povidone/SDS exhibit an improved in vitro dissolution rate for UPA compared with the micronized UPA, in particular when the SDS is present in an amount of 20% by weight.

However, slightly surprisingly, the solid dispersion UPA/povidone/SDS 3/6/1 is less effective than the solid dispersion UPA/povidone 3/7.

### 3. Crystallinity Profile of the UPA Present in the Solid Dispersions

The resulting solid dispersions were analyzed by X-ray powder diffractometry using the Phillips PW-1050 X Ray diffractometer device.

The diffraction spectrum of the solid dispersion UPA/Povidone/SDS 3/6/1 (see FIG. 3A) exhibits low-intensity diffraction peaks, suggesting that UPA is present in a predominantly amorphous form in the solid dispersion. Notably, this solid dispersion is stable since no significant conversion of the amorphous UPA to a crystalline form is observed after 1 month of storage at ambient temperature (see FIG. 3B).

On the other hand, an increase in the SDS content in the solid dispersion brings about the appearance of UPA in crystalline form, as attested to by the diffraction spectrum of the solid dispersion UPA/Povidone/SDS 3/5/2 (see FIG. 3B).

### Example 2

### Pharmaceutical Compositions Integrating a Solid Dispersion According to the Invention

Tables 3 and 4 hereinafter present examples of pharmaceutical compositions according to the invention. These pharmaceutical compositions can be obtained by mixing a solid dispersion according to the invention with various excipients, and then by shaping said mixture by direct compression so as to obtain tablets.

TABLE 3
Example of a composition according to
the invention comprising 5 mg of UPA

| | | % | |
|---|---|---|---|
| Ingredients | Function | by weight | mg/tablet |
| Solid dispersion | Active ingredient | 11.1 | 16.7 |
| UPA/povidone 3/7 | matrix | | (i.e. 5 mg of UPA) |
| Microcrystalline | Diluent | 54.4 | 81.6 |
| cellulose | | | |
| Mannitol | Diluent | 29.0 | 43.5 |
| Crospovidone | Disintegrant | 5.0 | 7.5 |
| Magnesium stearate | Lubricant | 0.5 | 0.75 |

| | |
|---|---|
| Total | 150 |

This composition can be used, for example, for the treatment of uterine fibromas.

TABLE 4
Example of a composition according to
the invention comprising 30 mg of UPA

| Ingredients | Function | % by weight | mg/tablet |
|---|---|---|---|
| Solid dispersion | Active ingredient | 48.0 | 100 |
| UPA/povidone 3/7 | matrix | | |
| Mannitol | Diluent | 48.0 | 100 |
| Crospovidone | Disintegrant | 3.6 | 7.5 |
| Magnesium stearate | Lubricant | 0.4 | 0.75 |

| | |
|---|---|
| Total | 208.25 |

This composition can be used, for example, in emergency contraception.

1. A solid dispersion comprising:
an active ingredient selected from the group consisting of selective progesterone receptor modulators, metabolites thereof and mixtures thereof, and
a pharmaceutically acceptable polymeric excipient.

2. The solid dispersion of claim 1, wherein the active ingredient is selected from the group consisting of 17α-acetoxy-11β-[4-N-methylaminophenyl]-19-norpregna-4,9-diene-3,20-dione, 17α-acetoxy-11β-[4-aminophenyl]-19-norpregna-4,9-diene-3,20-dione, ulipristal acetate and mixtures thereof.

3. The solid dispersion of claim 1, wherein the pharmaceutically acceptable polymeric excipient is selected from the group consisting of polyethylene glycols, N-vinyl-2-pyrrolidone polymers, N-vinyl-2-pyrrolidone copolymers, polyacrylic acid, polymethacrylic acid, copolymers based on acrylic acid, copolymers based on methacrylic acid, copolymers based on esters of methacrylic acid, cellulose, cellulose derivatives, and mixtures thereof.

4. The solid dispersion of claim 1, comprising:
an active ingredient selected from the group consisting of 17α-acetoxy-11β-[4-N-methylaminophenyl]-19-norpregna-4,9-diene-3,20-dione, 17α-acetoxy-11β-[4-aminophenyl]-19-norpregna-4,9-diene-3,20-dione, ulipristal acetate and mixtures thereof, preferably ulipristal acetate, and
a polymeric excipient selected from the group consisting of polyvinylpyrrolidones, N-vinyl-2-pyrrolidone copolymers and mixtures thereof.

5. The solid dispersion of claim 1, wherein weight ratio of the polymeric excipient to the active ingredient ranges from 1 to 50.

6. The solid dispersion of claim 1, further comprising a surfactant.

7. The solid dispersion of claim 6, wherein the weight ratio of the active ingredient to the surfactant ranges from 0.5 to 10.

8. The solid dispersion of claim 6, wherein the surfactant is a dodecyl sulphate salt.

9. The solid dispersion of claim 1, in the form of a deposit at the surface of a pharmaceutically acceptable carrier.

10. A method for preparing the solid dispersion of claim 1, comprising the steps of:
a) preparing a solution comprising the polymeric excipient and the active ingredient in a solvent, and
b) removing said solvent to obtain the solid dispersion.

11. A pharmaceutical composition comprising the solid dispersion of claim 1 and a pharmaceutically acceptable excipient.

12. The pharmaceutical composition of claim 11, comprising from 1 mg to 100 mg of active ingredient per dose unit.

13. The pharmaceutical composition of claim 11, suitable for oral administration.

14-15. (canceled)

16. The solid dispersion of claim 4, wherein the active ingredient is ulipristal acetate.

17. The solid dispersion of claim 8, wherein the surfactant is sodium dodecyl sulphate.

18. The pharmaceutical composition of claim 11, wherein the pharmaceutically acceptable excipient is selected from the group consisting of a diluent, a binder, a flow agent, a lubricant, a disintegrant and mixtures thereof.

19. The pharmaceutical composition of claim 12, comprising from 1 to 40 mg of active ingredient per dose unit.

20. The pharmaceutical composition of claim 13, in the form of a powder, a granule, a coated or uncoated tablet, or a gel capsule.

21. A method for providing contraception to a woman, comprising the step of administering the pharmaceutical composition of claim 11 to the woman.

22. A method for treating or preventing a gynecological disorder in a woman, comprising the step of administering the pharmaceutical composition of claim 11 to the woman.

23. A method for preparing the solid dispersion of claim 1, comprising the steps of:
a) preparing a mixture comprising the polymeric excipient and the active ingredient, wherein the polymeric excipient is in molten state, and
b) solidifying said mixture to obtain the solid dispersion.
